(19) United States
(12) Patent Application Publication
(10) Pub. No.: US 2004/0116676 A1
(43) Pub. Date: Jun. 17, 2004

(54) Methods for removal of contaminants from blood product solutions

(76) Inventors: JoAnn Hotta, Raleigh, NC (US); Marina N. Korneyeva, Raleigh, NC (US); Wytold Lebing, Clayton, NC (US)

Correspondence Address: WOMBLE CARLYLE SANDRIDGE & RICE, PLLC, P.O. BOX 7037, ATLANTA, GA, 30357-0037, US

(21) Appl. No.: 10/670,896

(22) Filed: Sep. 25, 2003

Related U.S. Application Data
(60) Provisional application No. 60/414,676, filed on Sep. 30, 2002.

(51) Int. Cl. C07K 16/00; A61M 37/00

(52) U.S. Cl. 530/390.1; 604/5.01

(57) ABSTRACT

A method of nanofiltration is provided, comprising passing a solution through at least one nanofiltration membrane having an average pore size of from about 15 nm to about 25 nm under normal flow conditions, wherein the solution components are sufficiently pure and at a concentration that allows the immunoglobulins to pass through at least one nanofiltration membrane. The solutions can contain, for example, immunoglobulins, Factor VIII, or plasmin/plasminogen.

This application claims benefit of U.S. Provisional Patent Application No. 60/414,676, filed Sep. 30, 2002.

### FIELD OF INVENTION

The invention relates to a filtration method for removal or reduction of contaminants in blood product preparations.

### BACKGROUND OF THE INVENTION

The removal of certain contaminants, especially viral contaminants, from blood products continues to be a major concern. Those involved in the production of such products continue to pursue methods for ensuring purity of blood products without impairing yield to a commercially unacceptable extent. In general, methods are sought that do not involve additional physical or chemical treatments that impair yield or introduce potentially harmful chemicals that may be difficult to remove completely from the product. Inactivation and/or removal of viral contamination continues to challenge the industry.

Filtration methods offer some significant advantages from a process perspective. However, their effectiveness, while satisfactory in specific circumstances, has not been demonstrated in several important areas. Many biotherapeutic proteins, such as intravenously administrable immunoglobulin (IGIV), Factor VIII, and plasminogen or plasmin, may be derived from human plasma or other sources known to contain viruses. One strategy to minimize the potential for virus transmission is to use manufacturing processes that remove and inactivate viruses while purifying the product. Unlike enveloped viruses, non-enveloped viruses, such as human parvovirus B19 and hepatitis A virus, are very difficult to inactivate or clear by current methods.

### SUMMARY OF THE INVENTION

The present invention provides methods that allow effective removal of small viral contaminants from solutions potentially contaminated by such viruses without significantly interfering with product yield or introducing any chemical agents that might be harmful or difficult to remove.

The invention relates to methods for removing contaminants from preparations that may contain viral contaminants by passing the solutions through at least one nanofiltration membrane under normal flow filtration conditions; and recovering the permeate solution.

In one aspect, the invention relates to nanofiltration of immunoglobulin preparations. The solutions containing the immunoglobulins are sufficiently pure and at a concentration that allows the immunoglobulins to pass through at least one nanofiltration membrane having an average pore size of from about 15 nm to about 25 nm.

In other aspects, the invention relates to nanofiltration of solutions containing Factor VIII or plasminogen in order to remove viral contamination.

### BRIEF DESCRIPTION OF THE INVENTION

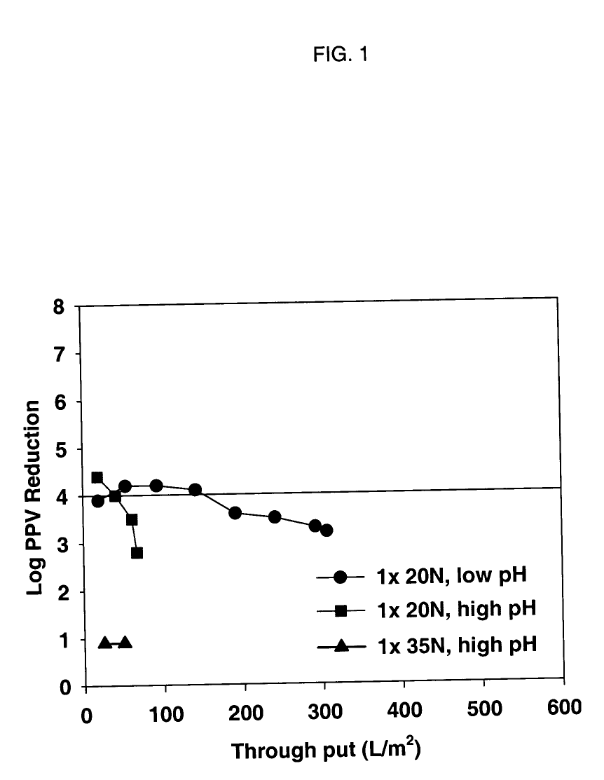

FIG. 1 is a graph showing the log reduction in porcine parvovirus (PPV) plotted against liters of throughput, showing pH effect on viral clearance for a single 20N (20 nm average pore size; PLANOVA 20N) filter used in normal flow mode. Results using a single 35N filter are shown for comparison.

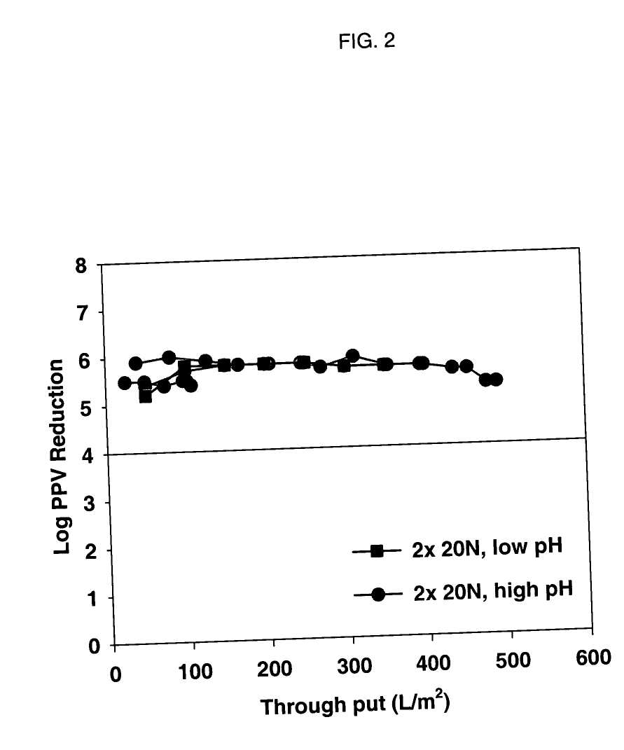

FIG. 2 is a graph showing the log reduction in PPV plotted against liters throughput, using two PLANOVA 20N filters.

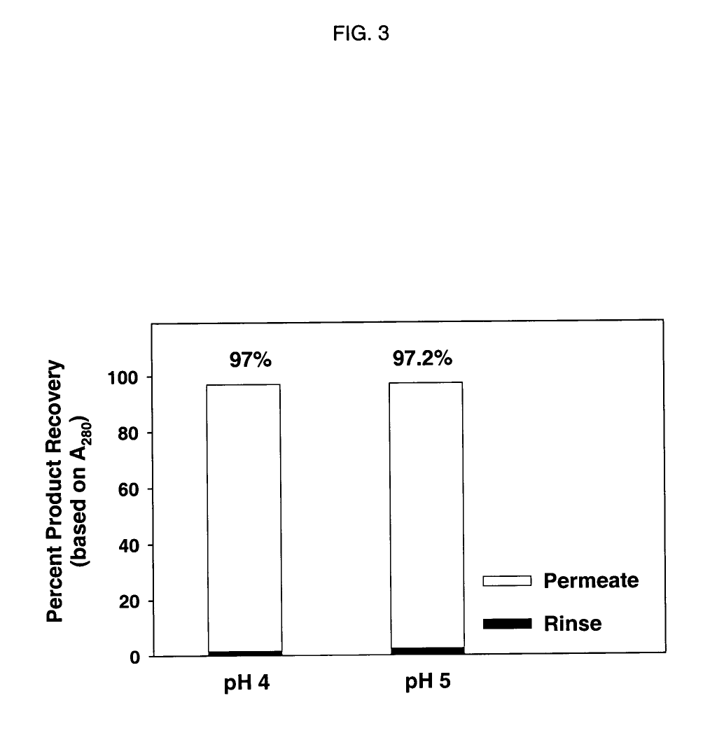

FIG. 3 is a graph showing the independence of IgG product recovery on pH.

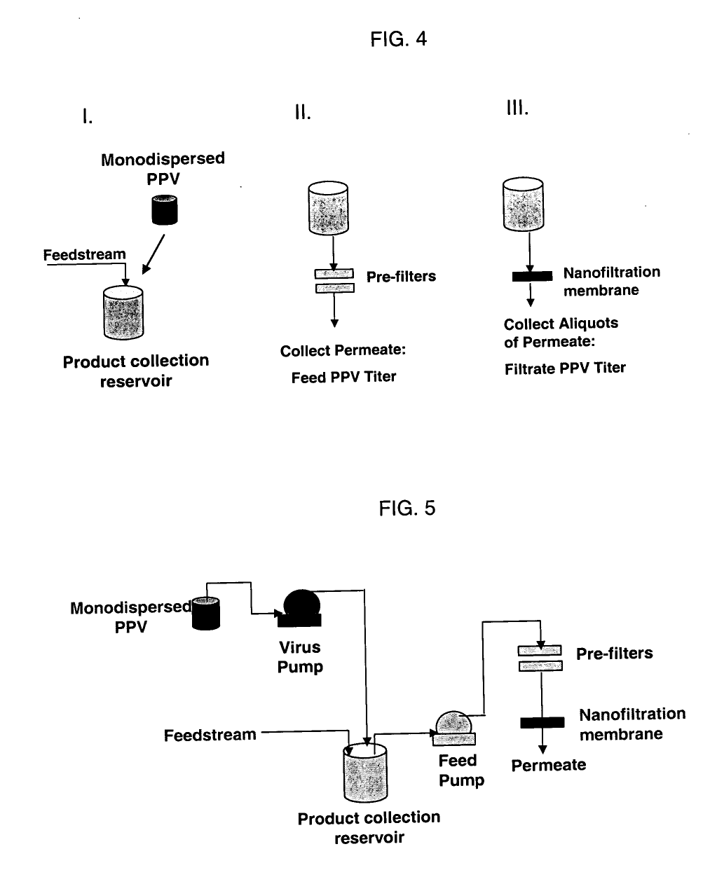

FIG. 4 is a schematic diagram of constant pressure nanofiltration according to one embodiment of the invention: I. illustrates collection of product and spiking with PPV; II. illustrates pre-filtering of spiked product and collection of permeate as nanofiltration feed; III. illustrates nanofiltration of product at constant pressure and collection of permeates for PPV titer and A₂₈₀.

FIG. 5 shows a schematic diagram of constant flow nanofiltration according to one embodiment of the invention: Nanofiltration product feedstream is illustrated in a “semi-continuous” mode with in-line pre-filtration to determine average flux and maximum throughput at acceptable pressure.

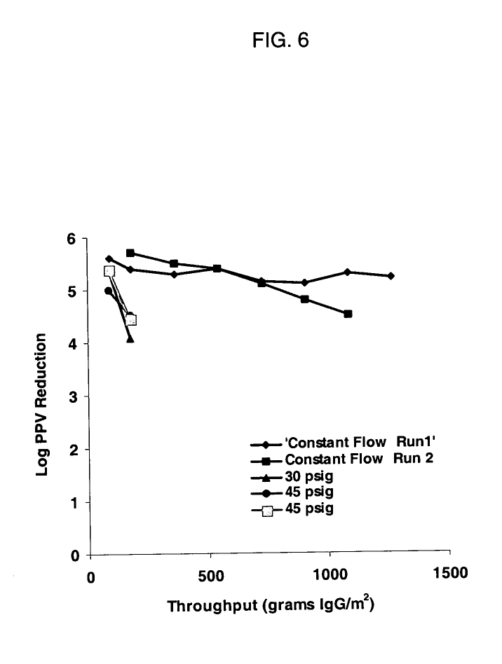

FIG. 6 is a graph showing the effect of constant pressure versus constant flow methods on clearance of PPV.

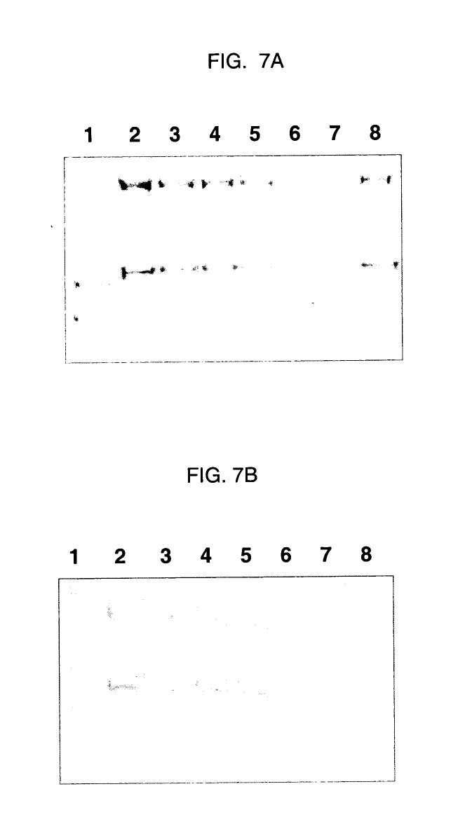

FIGS. 7A and 7B show polyacrylamide gel electrophoresis (PAGE) of fractions taken during nanofiltration of rFVIII, illustrating that the presence of high salt increases product recovery. Feed for experiments shown in FIG. 7B was made 250 mM in NaCl. Lanes shown are as follows: 1-markers; 2-Feed; 3-Permeate #1; 4-Permeate #2; 5-Permeate #3; 6-Rinse #1; 7-Rinse #2; 8-Retentate.

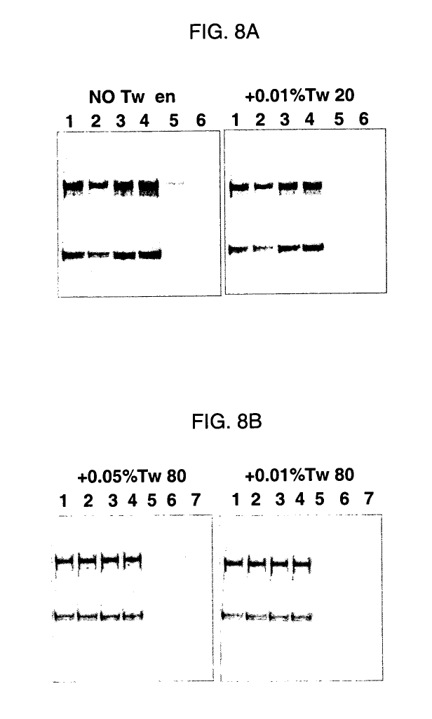

FIGS. 8A and 8B show PAGE of fractions taken during nanofiltration of rFVIII, illustrating that the addition of TWEEN increases flux and does not decrease yield. Lanes shown are as follows: FIG. 8A (using indicated concentrations of TWEEN 20), 1-Feed; 2-Permeate 1; 3-Permeate 2; 4-Permeate 3; 5-Rinse; 6-Retentate; FIG. 8B (using indicated concentrations of TWEEN 80), 1-Feed; 2-Permeate 1; 3-Permeate 2; 4-Permeate 3; 5-Rinse 1; 6-Rinse 2; 7-Retentate.

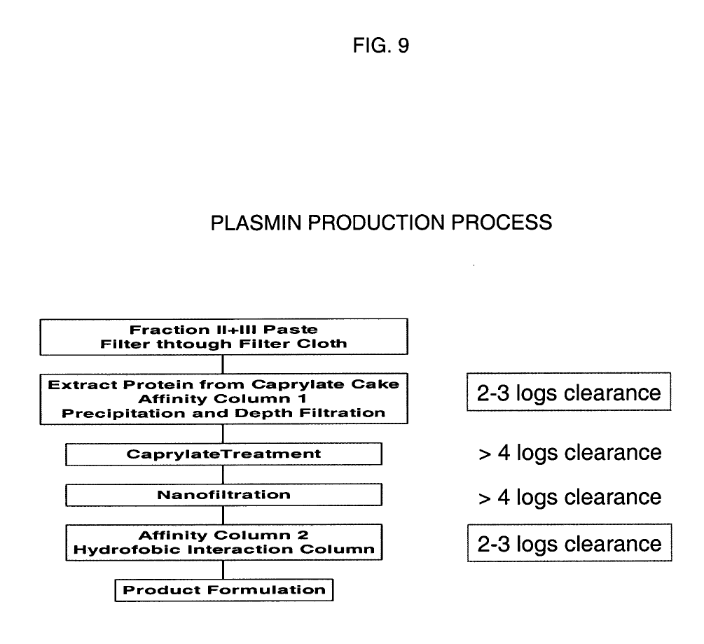

FIG. 9 shows a flow chart of a plasminogen production process. Viral clearance associated with particular steps is shown alongside the corresponding steps.

### DETAILED DESCRIPTION OF THE INVENTION

The present invention relates to nanofiltration of an immunoglobulin preparation that is characterized by the quality of being able to pass IgG through in the filter permeate without clogging the nanofiltration membrane. The invention also relates to nanofiltration of solutions containing recombinant Factor VIII (rFVIII) or plasminogen.

Unless expressly indicated to the contrary, the following terms have the meaning indicated below when used herein:

The terms “35N,” “20N,” “15N” indicate a filter membrane characterized by an average pore size of approximately 35 nm, 20 nm, or 15 nm, respectively.

The term “permeate” means the purified product which passes through the nanofiltration membrane.

The term “retentate” means material retained by the membrane.

The term “flux” means permeate flow rate per unit area of the membrane.

The invention relates to methods for removing contaminants from preparations that may contain viral contaminants by passing the solutions through at least one nanofiltration membrane under normal flow filtration conditions; and recovering the permeate solution.

In one aspect, the invention relates to nanofiltration of immunoglobulin preparations. The solutions containing the immunoglobulins are sufficiently pure and at a concentration that allows the immunoglobulins to pass through at least one nanofiltration membrane having an average pore size of from about 15 nm to about 25 nm.

In one embodiment, the solution containing immunoglobulin is greater than about 95% immunoglobulin. The solution containing immunoglobulin can be about 99% pure. The solution containing immunoglobulin can prepared in accordance with the methods disclosed in U.S. Pat. No. 5,886,154, incorporated herein by reference in its entirety.

In another embodiment, two nanofiltration membranes are used.

In another embodiment, the method includes prefiltering the solutions containing immunoglobulins by passing the solutions through a nanofiltration membrane having an average pore size of from about 30 nm to about 40 nm. The nanofiltration membrane can have an average pore size of about 35 nm.

In another embodiment, passing the solution through the at least one nanofiltration membrane under normal flow filtration conditions is performed under constant flow conditions.

In another aspect, the invention relates to nanofiltration of a solution containing Factor VIII in order to remove viral contamination using at least one nanofiltration membrane. The nanofiltration membrane(s) can have an average pore size of from about 15 nm to about 25 nm. Two nanofiltration membranes can be used. The Factor VIII can be produced recombinantly.

In another embodiment, the method further comprises prefiltering the solutions containing Factor VIII by passing the solution through a nanofiltration membrane having an average pore size of from about 30 nm to about 40 nm. The nanofiltration membrane used for prefiltration can have an average pore size of about 35 nm.

In another embodiment, the solution containing Factor VIII comprises a high salt buffer. The high salt buffer can have a conductivity of at least 20 mS/cm. The high salt buffer can also have a conductivity from about 20 to about 70 mS/cm. The high salt buffer can comprise about 250 mM NaCl.

In another aspect, the invention relates to nanofiltration of solutions containing plasminogen in order to remove viral contamination using at least one nanofiltration membrane. The nanofiltration membrane(s) can have an average pore size of from about 15 nm to about 25 nm. Two nanofiltration membranes can be used.

In one embodiment, the solution containing plasminogen can be at a pH of from about 2 to about 9. The pH can also be from about 3 to about 4. The pH can also be about 3.3.

Viral safety is an important prerequisite for all biotherapeutic products. While enveloped viruses are relatively easy to inactivate/clear by existing physical and chemical treatments, removal of non-enveloped viruses, such as human parvovirus B19 and hepatitis A virus, is much more difficult.

PLANOVA filters (Asahi Kasei America, Inc., Buffalo Grove, Ill.) were evaluated for their efficacy to remove porcine parvovirus (PPV) from a dilute IgG-containing solution. Normal flow filtration, at 12 psi constant trans-membrane pressure, of PPV-spiked IgG-containing material through one 35N filter resulted in little virus reduction. Filtration of the virus-spiked IgG solution through one 20N filter decreased the virus load by 3.5 log₁₀, while filtration of more than 500 L IgG per m², through two 20N filters in-series, resulted in 5.4 log₁₀ PPV reduction. Flow rates through two filters were the same as through one, and IgG recovery was greater than 97%. The data show nanofiltration through one or two 20 nm filters allows for efficient separation of virus, as small as parvovirus, from plasma-derived proteins as large as IgG (160 kD). See Examples 1 and 2, as well as FIGS. 1-3.

MILLIPORE NFP filters (Millipore Corp., Bedford, Mass.) were used under both constant pressure and constant flow conditions to evaluate parvovirus clearance from IgG solutions. The results indicated that both constant pressure and constant flow are effective; however, the constant flow mode allows for somewhat greater product recovery and semi-continuous, transparent inclusion of nanofiltration in the IgG preparation process. In addition, constant flow conditions allowed the consistent processing of a larger amount of IgG. See FIG. 6.

Similarly, nanofiltration can be used to purify Factor VIII according to the present invention. Recombinant Factor VIII (rFVIII) (KOGENATE, Bayer Corporation, Elkhart, Ind.) was spiked with PPV and subjected to nanofiltration using two PLANOVA 20N filters in normal flow mode. Greater than five log reduction in viral titer was achieved. Further, addition of the nonionic detergent TWEEN 20 increased flux and caused no decrease in yield. See FIGS. 8A and 8B.

Several nanofiltration membranes were found to be useful for clearance of PPV from plasminogen preparations. See Example 7 and Table 1, below. A combination of two PLANOVA 20N nanofilters in series provided optimal viral clearance and product recovery, although results achieved with certain other filters were noted as useful.

Having generally described the invention, the same will be more readily understood by reference to the following examples, which are provided by way of illustration and are not intended as limiting. Thus, the present invention is not to be limited in scope by the specific embodiments described herein, which are intended as single illustrations of individual aspects of the invention. Indeed, various modifications of the invention, in addition to those shown and described herein will become apparent to those skilled in the art from the foregoing description and accompanying drawings. Such modifications are intended to fall within the scope of the invention.

The entire disclosure of all publications (including patents, patent applications, journal articles, laboratory manuals, books, or other documents) cited herein are hereby incorporated by reference.

### EXAMPLES

Experiments were conducted to evaluate the feasibility of using nanofiltration to separate small viruses, such as porcine parvovirus (PPV), from large plasma derived proteins, such as IgG (160 kD). The goal was to achieve ≧4 log PPV reduction and ≧90% product recovery based on A₂₈₀. The test virus in these studies was porcine parvovirus (PPV), a small (18-24 nm) non-enveloped DNA virus with high resistance to physio-chemical agents. PPV is used generally to model human parvovirus B19.

It was noted that viral aggregates were present in crude PPV-spiked product solutions (confirmed by electron microscopy). Lower pH (pH 3 as opposed to pH 7) favored aggregation in crude preparations. Because of the possibility that viral aggregate could lead to overestimation of nanofilter capacity, monodispersed PPV, strain NADL-2, was obtained from Bioreliance Corporation, Rockville, Md.

Virus titers were quantitated by tissue culture infectious dose assays at 50% infectivity (TCID₅₀), using the method of Spearman and Kaerber (Spearman, C. and G. Kaerber, In: Bibrack, B. and G. Wittmann, eds., Virologische Arbeitsmethoden. Stuttgart: Fischer Verlag, pp. 37-39 (1974)). The virus reduction factor (RF) was determined by comparing the total virus in the input (Feed) to that of the output (Permeates+rinses).

RF=log total virus FEED−log total virus PERMEATE/RINSE

Nanofiltration of Immunoglobulin Preparations

IgG test solutions were generated from process intermediates obtained from a plasma fractionation facility (Bayer HealthCare, Inc., Clayton, N.C.) using a small scale model representative of production scale. Fraction II suspension (6-7% IgG) and Filtrate III (0.4-0.5% IgG) from the Cohn-Oncley fractionation procedure were evaluated (For the Cohn-Oncley fractionation procedure, see E. J. Cohn, et al., J. Amer. Chem. Soc., 68: 459 (1946); E. J. Cohn, U.S. Pat. No. 2,390,074; and Oncley, et al., J. Amer. Chem. Soc., 71:541 (1949), fully incorporated herein by reference).

Planova Filters

The PLANOVA filter module (Asahi Kasei America, Inc., Buffalo Grove, Ill.) consisted of a polycarbonate housing containing an assembly of hollow fibers that were spun from a cuprammonium cellulose solution. Each hollow fiber was 400 μm in diameter and the total effective membrane surface area of all fibers=0.001 m². The 35N and 20N filters were used to filter IgG. The ratings, 35N and 20N, refer to the mean pore size of the filters, as determined by the water flow rate method (e.g., 35N=35 nm pores, 20N=20 nm pores).

PLANOVA filters may be operated at constant transmembrane pressure in a normal flow filtration or tangential flow filtration mode. For these experiments only normal flow filtration, through one or two filters, at ≧12 psi was employed. PPV was spiked into an IgG test solution and the FEED was adjusted to a low pH (about 4) or high pH (about 5). After filtering the FEED, the membranes were rinsed with a sodium acetate buffer. Samples from the FEED, PERMEATES and RINSES were collected for virus titration.

### Example 1

### Effect of Average Pore Size and pH on Viral Clearance

No significant PPV reduction was achieved by filtering the IgG test solutions through a single 35N filter. Significant PPV reduction (≧4 log) was achieved, however, using a single 20N filter, but IgG capacity was dependent on pH and limited to 140 L/m². See FIG. 1.

### Example 2

### Effectiveness and Capacity of Two 20N Filters

IgG capacity through two filters was not limited by virus bleed through and was independent of pH. PPV clearance was 5-6 log even after filtering 500 L/m². During these experiments, the flow rates across the filters=48-60 L/m²/hr. Product recovery (total A₂₈₀ of the permeate+rinse compared to total A₂₈₀ of the feed), after filtration through two 20N filters, was independent of pH. Product recovery was ≧97% at both low and high pH.

Summary

No significant PPV reduction was achieved by filtering the IgG test solution through a single 35N filter. Nanofiltration of IgG through a single 20N filter was limited by virus bleed-through after filtering 140 L/m² and was dependent on low pH. Nanofiltration of 500 L/m² of IgG test solution, sequentially through two PLANOVA 20N filters, cleared significant levels (5-6 log) of PPV with little decrease in IgG yield (≧97% recovery based on A₂₈₀). Results obtained by nanofiltration through two filters were independent of pH, but the flow rate across the membranes was limited (48-60 L/m²/hr).

Fraction II suspension, which contained 6-7% IgG, could be filtered, but only 80-90% IgG was recovered. Filtrate III, which contained 16% EtOH, could not be filtered because EtOH caused the pores in the PLANOVA filters to shrink. pH was not an issue when two filters were used. However, when one filter was used, pH 4 was more effective than pH 5.

Millipore Viresolve NFP Filters

VIRESOLVE NFP (Normal Flow Parvovirus) filters (Millipore Corp., Bedford, Mass.) were tested to determine their usefulness for removal of parvovirus from IgG preparations, in accordance with the present invention. These filters comprise three layers of 180 kD membrane, and the manufacturer indicates that the filters will pass proteins up to 160 kD in size.

### Example 3

### Constant Pressure Nanofiltration

FIG. 4 is a schematic illustration of the protocol for evaluating constant pressure conditions for nanofiltration of IgG product using MILLIPORE NFP filters. Collection, prefiltration, and nanofiltration are illustrated: I. collect product and spike with PPV; II. pre-filter spiked product and collect permeate as the nanofilter feed; III. run product through the nanofilter at constant pressure and collect permeates for PPV titer and A₂₈₀. PPV clearance was similar (>4 log) between 30 psig and 45 psig constant operating pressures, although throughput was <300 grams IgG per m². See FIG. 6.

### Example 4

### Constant Flow Nanofiltration

FIG. 5 is a schematic illustration of the protocol for evaluating constant flow conditions for nanofiltration of IgG product using MILLIPORE NFP filters. IgG product feedstream was subjected to nanofiltration in a “semi-continuous” mode, with in-line pre-filtration to determine average flux and maximum throughput at acceptable pressure. Constant flow operation provided >4 log PPV clearance at >1000 grams IgG per m² with improved product recovery. See FIG. 6. Constant flow operating mode led to improved filtration performance and provided a better scale-down model.

Summary

Product recovery under constant pressure was from about 85% to about 98%. Under constant flow conditions, product recovery was from about 97% to about 104%. Recovery was assessed by A₂₈₀. Optimal operation required diluted protein concentration: Fraction II suspension at 6-7% IgG clogged the filters. Filtrate III, at 0.4-0.5% IgG, did not clog the filter, but protein recovery was limited to approximately 80-90%. PPV clearance was similar (>4 log) between 30 psig and 45 psig constant operating pressures, although throughput was <300 grams IgG per m².

Constant flow operation provided >4 log PPV clearance at >1000 grams IgG per m², with improved product recovery. Constant flow operating mode led to improved filtration performance and provided a better scale-down model.

Evaluation of the effect of pH showed that filtration at pH 4 was more effective than filtration at pH 5.

Nanofiltration of Recombinant Factor VIII

Preparations of recombinant human factor VIII (rFVIII) (KOGENATE®, Bayer Corporation, Elkhart, Ind.; see http://www.univgraph.com/bayer/inserts/kogenate.pdf, incorporated herein by reference) were spiked with PPV and subjected to nanofiltration to determine if such filtration could provide adequate reduction in viral titer.

### Example 5

### Presence of High Salt Increases rFVIII Recovery

Two PLANOVA filters (Asahi) were used in a normal flow filtration mode. PPV was spiked into an rFVIII test solution. Samples from the FEEDs, PERMEATEs and RINSEs were collected for virus titration.

For samples as shown in FIG. 7A, the feed buffer was 2.2% glycine, 1.1% sucrose, 20 mM NaCl, 20 mM histidine, and 5 mM CaCl₂; for samples shown in FIG. 7B, the feed buffer was the same except it was made 250 mM in NaCl. FIG. 7A shows product recovery of 70% by A₂₈₀; FIG. 7B shows 85-90% product recovery. Greater than 5 log of PPV reduction were shown in both experiments.

### Example 6

### Effect of TWEEN on Flux and Yield for rFVIII Nanofiltration

Experiments were conducted to determine the effect of the nonionic detergent TWEEN 20 on flux and yield of rFVIII during nanofiltration using PLANOVA 20N hollow fiber filters. The bioanalytical production filters were 4 m². TWEEN buffer concentrations and electrophoretic analysis of various filtration fractions were as shown in FIGS. 8A and 8B.

Product yield, calculated based on the Factor VIII potency assay of Barrowcliffe, varied only slightly from samples having 0.01% TWEEN 20, 0.05% TWEEN 80, and 0.01% TWEEN 80 (100, 99.8 and 99.9%, respectively, based on IU/ml measurements as described by Barrowcliffe, T. W., “Standardization of assays of factor VIII and factor IX,” Ric. Clin. Lab., 20(2):155-165 (1990)). Flux (L/hr/m²) was recorded as 36 (0.01% TWEEN 20), 48 (0.05% TWEEN 80), and 36 (0.01% TWEEN 80). For nanofiltration using no TWEEN, flux was 30 L/hr/m².

Nanofiltration of Pasminogen Preparations

Plasminogen was produced in accordance with the schematic diagram shown in FIG. 9. Additional details of the plasminogen production process steps can be found in published International Patent Application, Publication No. WO 01/36611.

### Example 7

### Nanofiltration of Plasminogen Preparation with Various Membranes

The results shown in Table 1 indicated that PLANOVA 15N nanofilter (Asahi) alone or coupled with PLANOVA 35N nanofilter, removed more than 4 log PPV. Also, nanofilters of larger pore size (PLANOVA 20N, PALL DV20 (Pall Corporation, East Hills, N.Y.), MILLIPORE NFP) can be combined in series to achieve higher viral clearance.

TABLE 1
PPV Clearance and Plasminogen Yield
Across Different Nanofilters

| | Log₁₀ TCID₅₀ | |
|---|---|---|
| | PPV | Protein |
| Filter Configuration | Reduction | Recovery |
| 2× Asahi 20 N | 4.6 | 99% |
| in series | | |
| NFP Millipore | 4.5 | 88% |
| (180 K-triple layer) | | |
| 2× PALL DV20 | 3.8 | 98% |
| in series | | |
| Asahi 35 N + 15 N | 4.5 | 90% |
| Asahi 15 N | 4.0 | 89% |

What is claimed is:

1. A method of nanofiltration of immunoglobulin preparations comprising passing a solution containing immunoglobulins through at least one nanofiltration membrane having an average pore size of from about 15 nm to about 25 nm under normal flow conditions, wherein the immunoglobulins are sufficiently pure and at a concentration that allows the immunoglobulins to pass through the at least one nanofiltration membrane.

2. The method of claim 1, wherein the solution containing immunoglobulin is greater than about 95% immunoglobulin.

3. The method of claim 1, wherein the solution containing immunoglobulin is about 99% pure.

4. The method of claim 1, wherein the solution containing immunoglobulins is passed through two nanofiltration membranes.

5. The method of claim 1, further comprising prefiltering the solutions containing immunoglobulins by passing the solutions through a prefiltration nanofiltration membrane having an average pore size of from about 30 nm to about 40 nm.

6. The method of claim 5, wherein prefiltration nanofiltration membrane has an average pore size of about 35 nm.

7. The method of claim 1, wherein passing the solution through the at least one nanofiltration membrane under normal flow filtration conditions is performed under constant flow conditions.

8. A method for reducing viral contamination that may be present in a solution containing Factor VIII, the method comprising passing the solution containing Factor VIII through at least one nanofiltration membrane under normal flow conditions and recovering the filtrate.

9. The method of claim 8, wherein the at least one nanofiltration membrane has an average pore size of from about 15 nm to about 25 nm.

10. The method of claim 8, wherein the solution containing Factor VIII is passed through two nanofiltration membranes.

11. The method of claim 8, wherein the Factor VIII is produced recombinantly.

12. The method of claim 8, further comprising prefiltering the solution containing Factor VIII by passing the solution through a prefiltration nanofiltration membrane having an average pore size of from about 30 nm to about 40 nm.

13. The method of claim 12, wherein the prefiltration nanofiltration membrane has an average pore size of about 35 nm.

14. The method of claim 8, wherein the solution containing Factor VIII comprises a high salt buffer.

15. The method of claim 14, wherein the high salt buffer has a conductivity of at least 20 mS/cm.

16. The method of claim 14, wherein the high salt buffer has a conductivity from about 20 to about 70 mS/cm.

17. The method of claim 14, wherein the high salt buffer comprises about 250 mM NaCl.

18. A method for reducing viral contamination that may be present in a solution containing plasminogen or plasmin, the method comprising passing the solution containing plasminogen or plasmin through at least one nanofiltration membrane under normal flow conditions, and recovering the filtrate.

19. The method of claim 18, wherein the nanofiltration membrane has an average pore size of from about 15 nm to about 25 nm.

20. The method of claim 18, wherein the solution containing plasminogen or plasmin is passed through two nanofiltration membranes.

21. The method of claim 18, wherein the solution containing plasminogen or plasmin is at a pH of from about 2 to about 9.

22. The method of claim 18, wherein solution containing plasminogen or plasmin is at pH of from about 3 to about 4.

23. The method of claim 18, wherein solution containing plasminogen or plasmin is at pH of about 3.3.

24. A method for reducing contaminants in solutions that may contain viral contaminants, the method comprising passing the solutions through at least one nanofiltration membrane under normal flow filtration conditions; and recovering the permeate solution, wherein contaminants that are reduced in the permeate relative to any amounts originally present in solution include non-enveloped viruses.

25. The method of claim 24, wherein the non-enveloped viruses are selected from the group consisting of human parvovirus B19 and hepatitis A virus.

26. A method of nanofiltration of immunoglobulin preparations comprising passing a solution containing immunoglobulins through at least one nanofiltration membrane having an average pore size of from about 15 nm to about 25 nm under normal flow conditions, wherein the immunoglobulins are sufficiently pure and at a concentration that allows the immunoglobulins to pass through the at least one nanofiltration membrane, wherein the solution containing immunoglobulins is prepared from a starting solution comprising immunoglobulins and other substances at an initial pH by
a) adding a source of caprylate ions to the starting solution and adjusting the pH to form a precipitate and a supernatant solution comprising immunoglobulins,
b) incubating the supernatant solution under conditions of time, pH, temperature, and caprylate ion concentration to inactivate substantially all viruses,
c) contacting the supernatant solution with at least one ion exchange resin under conditions that allow binding of at least some of the other substances to the resin while not allowing substantial binding of the immunoglobulins to the resin, and
d) collecting the antibodies.

27. A method of nanofiltration of immunoglobulin preparations comprising passing a solution containing immunoglobulins through at least one nanofiltration membrane having an average pore size of from about 15 nm to about 25 nm under normal flow conditions, wherein the immunoglobulins are sufficiently pure and at a concentration that allows the immunoglobulins to pass through the at least one nanofiltration membrane, wherein the solution containing immunoglobulins is prepared from a starting solution comprising immunoglobulins and other substances at an initial pH, by performing the sequential steps a) through e) of
a) adjusting the pH of the starting solution to be within a range of from about 3.8 to about 4.5 to form an intermediate solution comprising dissolved immunoglobulins,
b) adding a source of caprylate ions to the intermediate solution of step a) and adjusting the pH of the intermediate solution to be within a range of from about 5.0 to about 5.2 to form a precipitate and a supernatant solution comprising dissolved immunoglobulins,
c) incubating the supernatant solution under conditions of time, temperature, and caprylate ion concentration to inactivate substantially all enveloped viruses,
d) contacting the supernatant solution with at least one ion exchange resin under conditions that allow binding of at least some of the other substances to the resin while not allowing substantial binding of the immunoglobulins to the resin, and
e) collecting the immunoglobulins, the method further comprising the non-sequential step f) of,
f) separating the precipitate from the supernatant solution after at least one of steps b) or c) to result in a significant reduction of non-enveloped viruses.
